(19) United States
(12) Patent Application Publication
(10) Pub. No.: US 2023/0399270 A1
(43) Pub. Date: Dec. 14, 2023

(54) PENICILLIUM SIMPLICISSIMUM SP. NL-Z1 STRAIN FOR PROMOTING GROWTH OF ROOT NODULES AND INCREASING ABUNDANCE OF PROBIOTIC MICROORGANISM POPULATION

(71) Applicant: NANJING FORESTRY UNIVERSITY, Nanjing (CN)

(72) Inventors: JIAYAO ZHUANG, Nanjing (CN); CHAO LIU, Nanjing (CN); TONGXIN XU, Nanjing (CN); HAO YANG, Nanjing (CN); WENTAO ZHANG, Nanjing (CN)

(21) Appl. No.: 17/503,325

(22) Filed: Oct. 17, 2021

Related U.S. Application Data
(63) Continuation of application No. PCT/CN2021/087129, filed on Apr. 14, 2021.

(30) Foreign Application Priority Data
Aug. 25, 2020 (CN) 202010861282X

(51) Int. Cl. C05F 11/08; C12N 1/14

(52) U.S. Cl. CPC C05F 11/08; C12N 1/145; C12R 2001/80

(57) ABSTRACT

A Penicillium simplicissimum sp. nl-z1 strain for promoting growth of root nodules and increasing abundance of probiotic microorganism population is disclosed. The Penicillium simplicissimum sp. nl-z1 strain can promote Indigofera pseudotinctoria Matsum. production and nodule growth, and improve probiotic microorganisms and nutritional environment. After the application of Penicillium simplicissimum sp. nl-z1, the nutrient elements needed by plants such as potassium, calcium and magnesium in rock powder are effectively released, the fusion of spraying matrix and rock surface is promoted, the nutrition is continuously supplied for plants, the total nodule weight is increased by 152.63%, the content of hydrolyzed nitrogen is improved by 15.28%, and the symbiotic nitrogen fixation ability is effectively exerted. At the same time, the relative abundance of Mortierella in soil is increased from 12.81% to 94.95%, and the increasing amount of Mortierella and its promoting effect on plants are better than that of direct application of Mortierella.

### TECHNICAL FIELD

The present disclosure relates to the technical field of microorganism technology, and more specifically, to a Penicillium simplicissimum sp. NL-Z1 strain for promoting growth of root nodules and increasing abundance of probiotic microorganism population.

### BACKGROUND

Plant measures have become an important method for ecological restoration in barren areas such as bare rock slope. However, due to the limitation of site conditions, plants can not take root in this harsh ecological environment, and it is difficult to achieve long-term protection. In recent years, the application of microbial agents has received extensive attention. Microorganisms play an important role in mineral weathering, improving soil environment and regulating biological growth, which has a positive impact on ecological restoration. Leguminous trees and shrubs have always been popular species in ecological restoration, so the growth promotion mechanism of leguminous plants and the screening of high-quality rhizobia microbial agents have become the current research hotspot. Different rhizobia isolated from the root nodules of Leucaena leucocephala (Lam.) de Wit and different types of fertilizers were applied to crops, and the effects on crop growth, yield and nutrition were detected under field conditions, and finally, a high-quality Rhizobium radiobacter LB2 was screened. There are also studies on using biochar and inoculating rhizobia to improve the growth quality of Robinia pseudoacacia seedlings.

However, these high-quality growth promoting bacteria do not necessarily adapt to the extreme ecological environment in the restoration area. The microorganisms living in the extreme environment show special adaptability, so that they can thrive in such an environment. Therefore, whether some functional microorganisms can be screened out from extreme environment, such as phosphate solubilizing function, and then the screened microorganisms can be used in leguminous plants growth promotion research to further explore the growth promotion function and mechanism, rather than limited to the screening of high-quality rhizobia.

If there are such microorganisms, they can not only ensure their adaptability to extreme ecological environment, but also realize their important role in ecological restoration.

In addition, there are more insoluble phosphates in soil environment, but less available phosphorus that can be absorbed and utilized by plants. Insoluble phosphates can be transformed into soluble phosphates for plant absorption and utilization under the decomposition of phosphate solubilizing microorganisms, which can increase the utilization rate of phosphate fertilizer in soil and avoid environmental pollution caused by excessive use of phosphate fertilizer.

Therefore, it is an urgent problem to be solved for those skilled in the art to find a kind of microorganism which has long-term restoration effect from the extreme environment to be used in the poor ecological restoration area, so as to study their ability to promote mineral dissolution and plant growth, and clarify the evolution of soil fungal community structure and its relationship with soil physical and chemical properties under the condition of long-term application of microbial agents, so as to provide theoretical basis, practical guide and basis for application of bacteria for revegetation of rocky desertification such as bare rock walls.

### SUMMARY

In view of this, the present disclosure provides a Penicillium simplicissimum sp. NL-Z1 strain which can promote the growth of root nodules and increase the abundance of probiotic microorganism population.

Penicillium simplicissimum sp. NL-Z1 is deposited in China Center for Type Culture Collection, Wuhan University, China. The deposit number is CCTCC NO: M2019999. The deposit date is Dec. 3, 2019.

The present disclosure selects highly efficient phosphate solubilizing bacteria from bare rock slope rock, and further studies their ability to promote mineral dissolution and plant growth. High throughput sequencing technology is used to study the evolution of soil fungal community structure and its coupling relationship with soil physical and chemical properties. The results of this study will provide theoretical basis and practical guidance for the revegetation of rocky desertification such as bare rock walls, and provide a beneficial basis for the application of bacteria.

In order to achieve the above purpose, technical solutions of the present disclosure are specifically described as follows.

In a first aspect, the disclosure provides a method for promoting root nodule growth. Penicillium simplicissimum sp. NL-Z1 is used as a bacterial fertilizer, and the deposit number is CCTCC NO: M2019999.

In a second aspect, the disclosure provides a method for improving a relative abundance of microorganisms. Penicillium simplicissimum sp. NL-Z1 is used as a bacterial fertilizer, and the deposit number is CCTCC NO: M2019999.

In a third aspect, the disclosure provides an application of Penicillium simplicissimum sp. NL-Z1 in promoting plant production of Indigofera pseudotinctoria Matsum. Penicillium simplicissimum sp. NL-Z1 is used as a bacterial fertilizer, and the deposit number is CCTCC NO: M2019999.

Preferably, the nodule weight of Indigofera pseudotinctoria Matsum. seedlings treated with Penicillium simplicissimum sp. NL-Z1 is increased significantly. The total nodule weight is increased at least 152.63%, and the bioactive enzymes is increased from 45 to 121 and is increased by 169%.

Preferably, after a treatment with strain NL-Z1, the abundance of probiotic microorganism population is increased. Ascomycota is decreased from 85.32% to at least 1.32%. Mortierellomycota is increased from 12.81% to at least 94.95%, becoming a dominant phylum after the treatment with strain NL-Z1.

Preferably, the Indigofera pseudotinctoria Matsum. seedlings treated with strain NL-Z1 can promote the growth of plant roots and aboveground parts due to the increase of nodule weight, enzyme activity and the abundance of probiotic microorganism population. A root biomass is at least 1.60 g, increased by 33.33%. A root area is 246.53 cm², increased by at least 6.14%. A root volume is 2.12 cm³, increased by at least 23.98%. An aboveground biomass is 7.90 g, increased by 37.63%. An average ground diameter is 5.02 mm, increased by 10.33%. An average height of aboveground parts is at least 74.67 cm, increased by 36.58%. An average leaf area is at least 6.25 cm², increased by 24.50%. A hydrolytic nitrogen of rhizosphere soil increased by at least A soil pH is decreased from 7.06 to 6.79.

The beneficial effect is that the root nodules of the Indigofera pseudotinctoria Matsum. seedlings treated with strain NL-Z1 can be better biological nitrogen fixation, so that the nitrogen in the plant can be supplemented, which is conducive to its own growth and development. A root biomass is at least 1.60 g, increased by 33.33%. A root area is 246.53 cm², increased by at least 6.14%. A root volume is 2.12 cm³, increased by at least 23.98%. An aboveground biomass is 7.90 g, increased by 37.63%. An average ground diameter is 5.02 mm, increased by 10.33%. An average height of aboveground parts is at least 74.67 cm, increased by 36.58%. An average leaf area is at least 6.25 cm², increased by 24.50%. A hydrolytic nitrogen of rhizosphere soil increased by at least 15.28%. And the soil has a certain degree of acidification, and the soil pH is decreased from 7.06 to 6.79. Strain NL-Z1 can transform the nitrogen in soil into the form that can be directly absorbed and utilized by plants, so as to promote the growth of plants and create a favorable environment for the growth of Indigofera pseudotinctoria Matsum. After a treatment with strain NL-Z1, Ascomycota is decreased from 85.32% to at least 1.32%, and Mortierellomycota is increased from 12.81% to at least 94.95%, becoming a dominant phylum after the treatment with strain NL-Z1.

It can be seen from the above technical scheme that, compared with the prior art, the disclosure provides a Penicillium simplicissimum sp. NL-Z1 strain which can promote the growth of root nodules and improve the abundance of probiotic microorganism population. The obtained technical effect is that after the application of Penicillium simplicissimum sp. NL-Z1 provided by the disclosure, the nutrient elements needed by plants such as potassium, calcium and magnesium in rock powder can be effectively released, the fusion of spraying matrix and rock surface can be promoted, and the nutrition for plants can be continuously supplied. In particularly, Penicillium simplicissimum sp. NL-Z1 promotes nitrogen fixation of Indigofera pseudotinctoria Matsum., increases total nodule weight, root biomass, ground diameter, seedling height and average leaf area, and increases the relative abundance of Mortierella in soil, which effectively exerted the symbiotic nitrogen fixation ability. Moreover, the increase of relative abundance of Mortierella and its promoting effect on plant growth are better than that of direct application of Mortierella. The disclosure also shows the evolution relationship of soil fungal community structure under the condition of long-term application of microbial agent and the coupling relationship with soil physical and chemical properties through high throughput sequencing technology, which provides theoretical basis and practical guidance for the revegetation of rocky desertification such as bare rock walls, and provides a beneficial basis for the application of bacteria.

### BRIEF DESCRIPTION OF THE DRAWINGS

In order to explain the embodiments of the present disclosure or the technical solutions in the prior art more clearly, the following drawings that need to be used in the description of the embodiments or the prior art will be briefly introduced. Obviously, the drawings in the following description are only embodiments of the present disclosure. For those of ordinary skill in the art, other drawings can be obtained based on the drawings disclosed without creative work.

FIG. 1 is the schematic diagram of the strains to be screened to promote the release of available phosphorus and pH in rock powder provided by the disclosure, in which the sequence from left to right is CK, X-4, X-8, X-11, x-14 and NL-Z1.

FIG. 2 is the schematic diagram of the change of potassium release by the strains to be screened provided by the disclosure, which are X-4, X-8, X-11, X-14, NL-Z1 and CK in sequence.

FIG. 3 is the schematic diagram of the change of calcium release by the strains to be screened provided by the disclosure, which are X-4, X-8, X-11, X-14, NL-Z1 and CK in sequence.

FIG. 4 is the schematic diagram of the change of magnesium release by the strains to be screened provided by the disclosure, which are X-4, X-8, X-11, x-14, NL-Z1 and CK in sequence.

FIG. 5 is the schematic diagram of BLAST comparison provided by the disclosure.

FIG. 6 is the schematic diagram of available phosphorus, hydrolytic nitrogen concentration and pH change of potted plants treated by the strain NL-Z1 provided by the disclosure.

FIG. 7 is the schematic diagram of the microbial community composition at phylum level in the control group and the potted soil treated by the strain NL-Z1 provided by the disclosure.

FIG. 8 is the schematic diagram of the species composition at genus level in the control group and the potted soil treated by the strain NL-Z1 provided by the disclosure.

FIG. 9 is the schematic diagram of species difference significance test at genus level by using Student's T test method provided by the disclosure.

FIG. 10 is the schematic diagram of the relationship among environmental factors, flora and samples provided by the disclosure.

### DETAILED DESCRIPTION OF EMBODIMENTS

Technical solutions of the present disclosure will be clearly and completely described below with reference to the drawings in the embodiments. Obviously, the described embodiments are only part of the embodiments of the present disclosure, not all of them. Other embodiments made by those skilled in the art without sparing any creative effort should fall within the scope of the disclosure.

The embodiments disclose a Penicillium simplicissimum sp. NL-Z1 strain for promoting growth of root nodules and increasing abundance of probiotic microorganism population.

The raw materials and reagents involved in the embodiment were obtained through commercial channels, and there was no requirement for their brands. The methods not mentioned were commonly used in experiments. For example, Excel software was used for data processing and sequencing registration, SPSS software was used for statistical analysis, R language (ggplot2 package and vegan package) was used for microbial community structure map and RDA map, and STAMP software was used for species difference analysis and mapping. It will not be repeated here.

### Embodiment 1

1 Source of Sample

The strains were screened from rock samples from the bare rock slope of Yueyang Avenue, Yueyang City, Hunan Province, China. Rock samples were collected from the upper, middle and lower parts of the slope and taken back to the laboratory for processing, which were used for the composition analysis of mineral samples and subsequent tests. According to the results of mineral analysis, the main compositions of rock samples are as follows: K₂O 3.71%, Na₂O 1.39%, CaO 0.21%, MgO 1.28%, P₂O₅ 0.11%, Fe₂O₃ 6.81%, Al₂O₃ 15.21%, MnO 0.04%.

2 Isolation and Screening

2.1 Medium
(1) Isolation medium: NaCl 0.3 g, KCl 0.3 g, (NH₄)SO₂ 0.5 g, MgSO₄·7H₂O 0.3 g, FeSO₄·7H₂O 0.03 g, MnSO₄·4H₂O 0.3 g, Ca₃(PO₄)₂ 5.0 g, sucrose 10 g, agar 15-20 g, deionized water 1000 ml, pH 7.0-7.5.
(2) PDA solid medium: potato 200 g, glucose 20 g, agar 18 g, water 1000 ml, pH 7.0-7.2.
(3) Beef extract peptone medium: beef extract 3 g, peptone 10 g, NaCl 5 g, agar 20 g, deionized water 1000 ml, and pH7.0-7.2.
(4) Mungina inorganic phosphorus medium: glucose 10 g, (NH₄) SO₂ 0.5 g, NaCl 0.3 g, KCl 0.3 g, MgSO₄·7H₂O 0.3 g, FeSO₄·7H₂O 0.03 g, MnSO₄ 0.03 g, Ca₃(PO₄) 25.0 g, agar 20 g, deionized water 1000 ml, pH 7.0-7.5.
(5) Mungina organic phosphorus medium: glucose 10 g, (NH₄)SO₂ 0.5 g, NaCl 0.3 g, KCl 0.3 g, MgSO₄·7H₂O 0.3 g, FeSO₄·7H₂O 0.03 g, MnSO₄ 0.03 g, CaCO₃ 5.0 g, lecithin 0.3 g, agar 20 g, deionized water 1000 ml, pH 7.0-7.5.
(6) Modified Mungina medium: the phosphorus containing drugs in (3) or (4) were replaced by mineral samples.
(7) LB liquid medium: peptone 10 g, yeast extract 5 g, NaCl 5 g, deionized water 1000 ml, pH 7.2.

2.2 Screening

The isolated single strain was activated and cultured in the plates of the Mungina organic phosphorus medium and the Mungina inorganic phosphorus medium. Three parallel experiments were conducted for each strain. They were cultured in 28° C. incubator for 5 days with organic phosphorus and 7 days with inorganic phosphorus. The phosphate solubilizing bacteria were the ones with transparent phosphate solubilizing circle in the plates. The colony diameter d and transparent circle diameter D were measured respectively, and the ratio D/d of the transparent circle diameter D and the colony diameter d was calculated, so as to judge the phosphate solubilizing ability of the phosphate solubilizing bacteria.

As shown in Table 1, a total of 21 strains with phosphate solubilizing effect were isolated from the rocks, including 20 strains of phosphate solubilizing bacteria and 1 strain of phosphate solubilizing fungi. Finally, five phosphate solubilizing bacteria and fungi with good effect were selected for further study, which were X-4, X-8, X-11, X-14 and NL-Z1.

TABLE 1
D/d statistics of phosphate solubilizing
effect of phosphate solubilizing bacteria
Unit: cm

| | D/d | D/d |
|---|---|---|
| Name | (organic) | (inorganic) |
| X-4 | 3.61a | 2.13c |
| X-8 | — | 2.60bc |
| X-11 | 3.06ab | 4.54a |
| X-14 | 3.47a | 2.81b |
| X-17 | 2.22c | — |
| X-19 | 2.09cd | 2.60bc |
| X-25 | 2.49b | 1.53d |
| X-27 | 2.50b | 2.00c |
| X-30 | — | 1.69d |
| X-33 | — | 1.50d |
| X-34 | — | 1.44df |
| X-35 | — | 1.27f |
| X-38 | 2.41bc | 1.93c |
| X-42 | — | 1.57d |
| X-43 | 2.83b | — |
| X-44 | 1.42e | — |
| X-48 | 2.25c | — |
| X-53 | 1.79d | — |
| X-55 | 2.22c | 1.37f |
| X-58 | 2.17c | — |
| NL-Z1 | 3.75a | 1.55d |

2.3 Dissolution test of rock powder

100 ml conical flasks were used. Each conical flask was filled with 30 ml of modified Mungina liquid medium and 1.5 g of 200 mesh rock powder. The selected phosphate solubilizing bacteria were made into seed liquid, and 3% of the liquid volume was added into each flask. The non-inoculated bacteria were used as blank control, and each treatment was conducted in three parallel. The culture was carried out at 30° C. and 160 rpm. The pH of fermentation broth was determined on the 4th, 7th and 10th day of the experiment. Secondly, the fermentation broth was centrifuged to extract the supernatant, the content of available phosphorus was determined by Mo—Sb anti-colorimetric method, and the ion content of potassium, calcium and magnesium was determined by atomic absorption spectrometry.

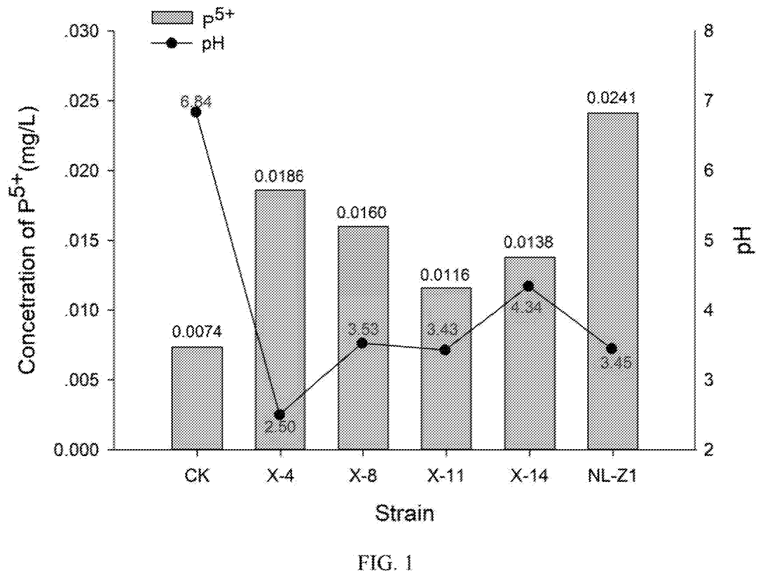

The results showed that according to FIG. 1, the five strains (X-4, X-8, X-11, X-14 and NL-Z1) all promoted the release of available phosphorus in rock powder to a certain extent, and the strain NL-Z1 had the highest peak release of available phosphorus, with the concentration of 0.0241 mg/L, which was 3.26 times of the control group. At the same time, the pH value of fermentation broth was measured, and the pH value of each treatment group decreased. Therefore, it is speculated that the acidolysis of the strain is an important mechanism for the dissolution of rock. The strain can improve the dissolution of trace elements in rock powder by secreting a large number of acidic substances.

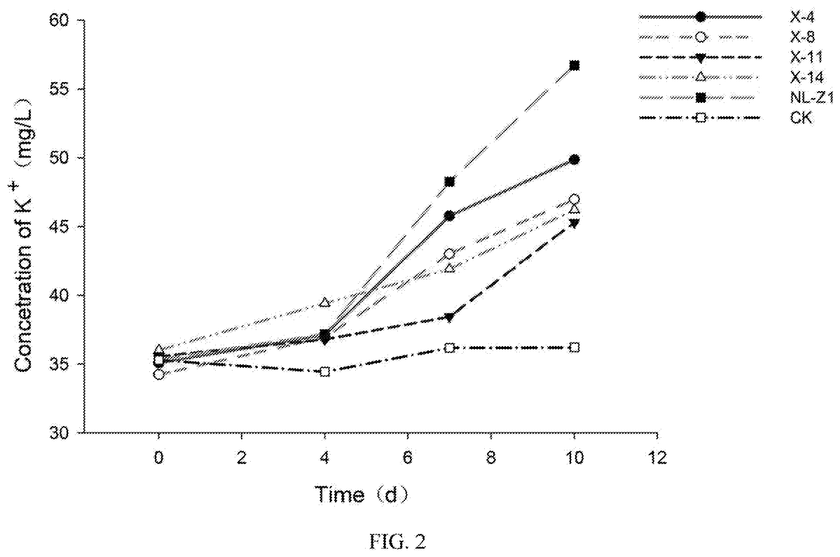

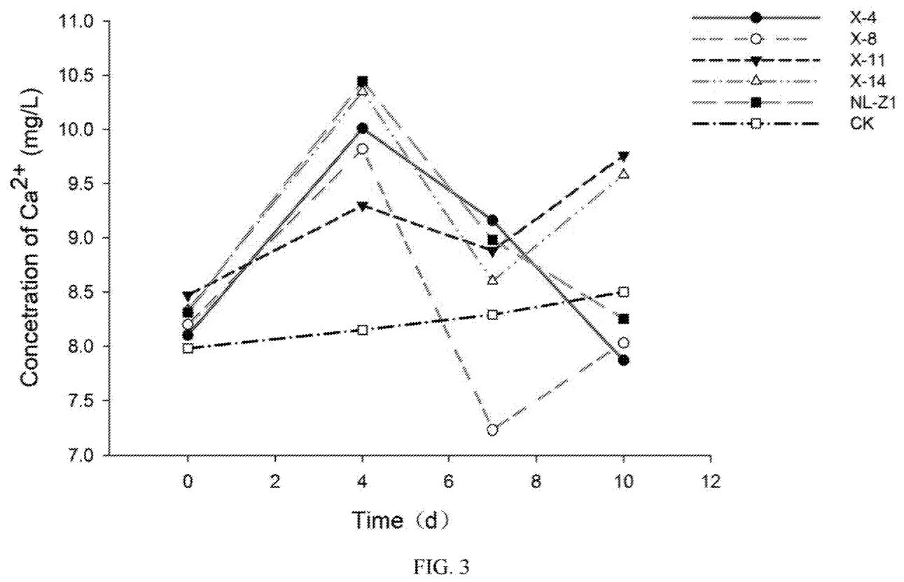

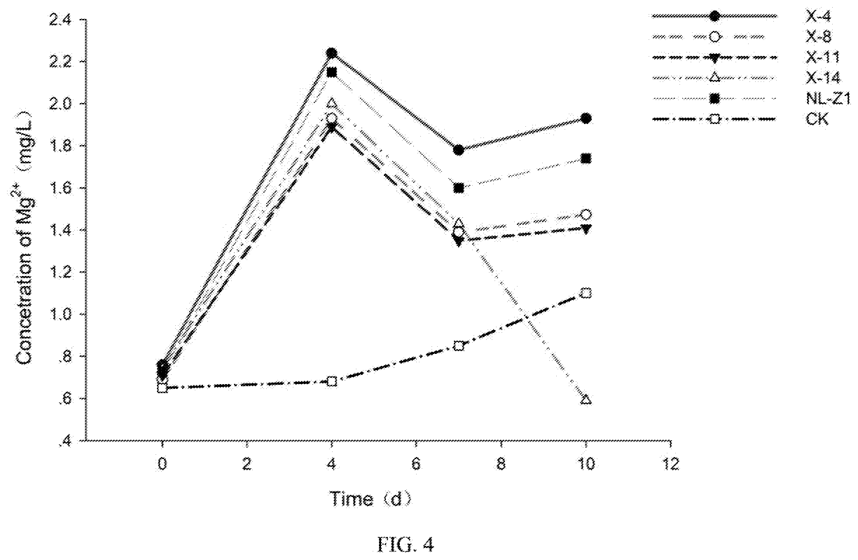

FIGS. 2-4 show the dynamic changes of the release of potassium, calcium and magnesium from rocks by different strains. Compared with the control, fungus NL-Z1 had a stronger ability to release various elements, and its release peaks of potassium, calcium and magnesium increased by 29.07%, 23.65% and 95.45% respectively. In general, the release of P, K, Ca and Mg from the rock powder showed an upward trend at first, and then a downward trend. When the strain was in the growth stage, the concentration of elements in the fermentation broth continued to increase. With the progress of the experiment, because of the use of a large number of nutrients in the fermentation broth by the strain in the growth stage and the limitation of fermentation space, the element dissolution rate was less than the utilization rate, showing a downward trend. According to the dynamic changes of each element, the strain NL-Z1 had a good release effect for each element, and compared with other strains, the strain NL-Z1 could effectively promote the dissolution of rocks.

The strain NL-Z1 was sent to Shanghai Jinyu Medical Laboratory Center for its gene sequence identification.

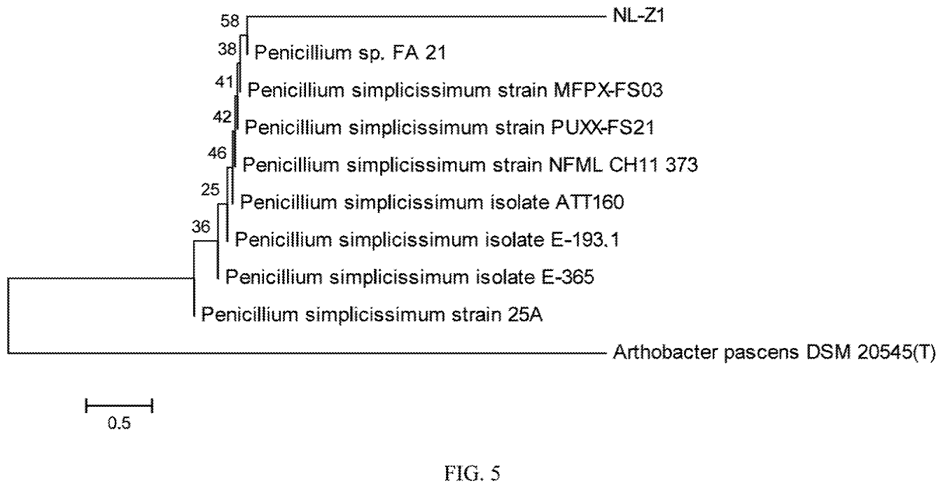

Compared with Penicillium simplicissimum by BLAST, the similarity reached 99.09%. FIG. 5 shows the constructed phylogenetic tree, and NL-Z1 was determined to be Penicillium sp. FA 21 by the analysis of the phylogenetic tree.

NL-z1 was deposited in China Center for Type Culture Collection with the deposit number of CCTCC No: M2019999.

In embodiment 1, the test soil samples were screened by using the method of plate screening of Mungina organic (inorganic) phosphorus medium. The phosphorus solubilizing circle and the available phosphorus content in the fermentation broth on the Mungina plate can only preliminarily explain the ability of the strain to solubilize phosphorus, and it is unable to evaluate the phosphorus solubilizing and other technical effects of the phosphate solubilizing bacteria more reliably. Therefore, the dissolution test of rock powder was carried out by replacing the phosphorus content of Mungina medium with the sample rock powder in embodiment 1. The dissolution capacity of the strain was judged by analyzing the change of phosphorus in the fermentation broth. The results showed that NL-Z1 could release the nutrients needed by plants such as phosphorus, potassium, calcium and magnesium in rock powder effectively, which indicated that the strain had the effect of promoting the fusion of spray seeding matrix and rock surface, and ensuring the sustainable nutrition supply of plants.

### Embodiment 2

The effect of bacteria on promoting growth was explored. Combined with pot experiment, through planting Indigofera pseudotinctoria Matsum. and observing its growth, the screened strains were tested more comprehensively and closer to the actual application, and the potential effects in the actual environmental restoration and production application were explored.

The Preparation of the Agent Containing NL-Z1:

After the strain was activated, it was inoculated into the liquid medium and fermented for 3 days. The OD600 was measured by ultraviolet spectrophotometer. The OD600 value of the bacteria solution was kept in the range of 0.8-1.2 by dilution or continuous fermentation, and then sealed and stored in the refrigerator at 4° C. for standby.

When applying bacteria in pot, the stored bacteria solution was diluted 100 times, and 60 ml diluted bacteria solution was put into each pot. Three parallel treatments were set for each treatment, and the sterile medium was used as blank control.

Seedling Planting of Indigofera pseudotinctoria Matsum.:

The leguminous plant Indigofera pseudotinctoria Matsum. was selected as the experimental object in pot experiment. The seeds were sterilized with sodium hypochlorite, and then the robust buds were selected for planting. The soil used for potting was provided by Jiangsu Xingnong Matrix Technology Co., Ltd. Three young buds were put into each pot, and the seedlings were thinned after one month's growth. One robust seedling was kept in each pot (the growth was consistent with each pot), and the prepared bacterial agent was applied.

Determination and Method of Potted Plant Index:

For plants: vernier caliper and tape were used to measure the ground diameter and seedling height; leaf area and root morphology were measured by root scanner (10 upper, middle and lower leaves were selected for each pot); the number of nodules was recorded and the plants were dried to determine the aboveground and underground biomass.

For potted soil: the pH value was determined by a mettler Toledo pH meter (the ratio of soil to water was 5:1); the effective phosphorus in soil was determined by acid soluble Mo—Sb anti-colorimetric method; the hydrolytic nitrogen in soil was determined by alkali-hydrolyzed diffusing method.

Effects on the Growth of Underground Part of Indigofera pseudotinctoria Matsum.:

As shown in Table 2, the average nodule number in the sterile treatment group was 8, and the total nodule weight was 0.19 g. The average nodule number in the strain NL-Z1 treatment group with was 6, and the total nodule weight was 0.48 g. Compared with the control, the nodule number in the seedlings treated with NL-Z1 was slightly lower, but the total nodule weight increased by 152.63% (P<0.05). Leguminous plants can be biological nitrogen fixation, so that the nitrogen in the plant can be supplemented, which is conducive to their own growth and development. The root biomass, root area and root volume of sterile treatment group were 1.20 g, 232.26 cm² and 1.71 cm³, respectively. The root biomass of the strain NL-Z1 treatment group was 1.60 g, significantly increased by 33.33% (P<0.05); the root area was 246.53 cm², increased by 6.14%; and the root volume was 2.12 cm³, increased by 23.98%.

TABLE 2
Effect of strain NL-Z1 on the root of Indigofera pseudotinctoria Matsum.

| Experi- | Dry | Root | Root | | Total |
|---|---|---|---|---|---|
| mental | weight | area | volume | Nodule | nodule |
| treatment | (g) | (cm2) | (cm3) | number | weight (g) |
| CK | 1.20 ± 0.07b | 232.26 ± 8.78a | 1.71 ± 0.02a | 7.67 ± 4.04a | 0.19 ± 0.06b |
| NL-Z1 | 1.60 ± 0.06a | 246.53 ± 25.69a | 2.12 ± 0.21a | 6.00 ± 2.00a | 0.48 ± 0.11a |

Effects on the Growth of Aboveground Part of Indigofera pseudotinctoria Matsum.:

The aboveground growth of Indigofera pseudotinctoria Matsum. seedlings was shown in Table 3. The aboveground indexes of the strain NL-Z1 treatment group were higher than those of the sterile treatment group. The average aboveground biomass of the strain NL-Z1 treatment group was 7.90 g, significantly increased by 37.63% (P<0.05). The average ground diameter was 5.02 mm, significantly increased by 10.33% (P<0.05). The average plant height was 74.67 cm, increased by 36.58%. The average leaf area was 6.25 cm², significantly increased by 24.50% (P<0.05).

TABLE 3
Effect of strain NL-Z1 on the aboveground part of
Indigofera pseudotinctoria Matsum.

| | | | | Average |
|---|---|---|---|---|
| Experi- | Dry | Ground | Plant | leaf |
| mental | weight | diameter | height | area |
| treatment | (g) | (mm) | (cm) | (cm2) |
| CK | 5.74 ± 0.07b | 4.55 ± 0.39b | 54.67 ± 2.52b | 5.02 ± 0.30b |
| NL-Z1 | 7.90 ± 0.90a | 5.02 ± 0.10a | 74.67 ± 5.03a | 6.25 ± 0.13a |

Effects of the Strain NL-Z1 on Physicochemical Properties of Potted Soil:

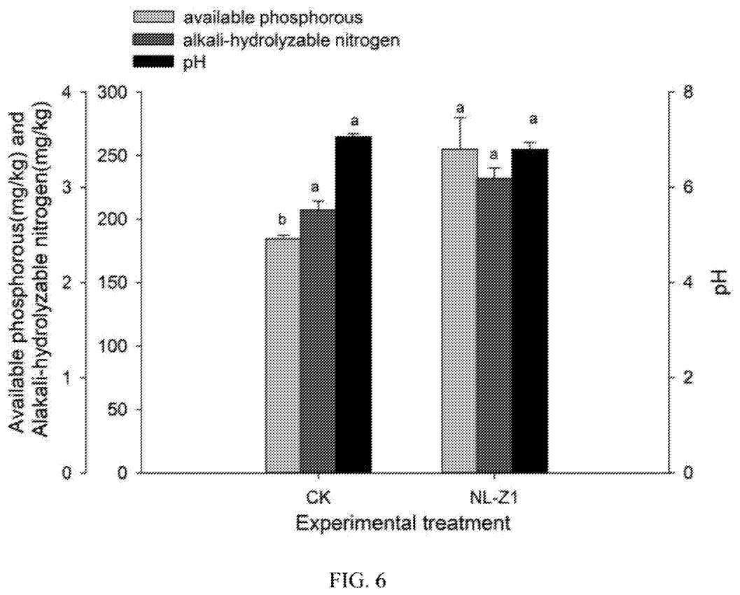

According to FIG. 6, the concentration of available phosphorus and hydrolyzed nitrogen in potted plant treated by strain NL-Z1 was 3.360 mg/kg and 238.83 mg/kg respectively, and the content of available phosphorus increased by 36.59% (P<0.05), and the hydrolysate nitrogen increased by 15.28%. The pH of potted soil decreased from 7.06 to 6.79. The pot experiment further confirmed that strain NL-Z1 could transform the phosphorus and nitrogen in soil into a form that could be absorbed and utilized directly, thus promoting the growth of plants and creating an environment conducive to the growth of Indigofera pseudotinctoria Matsum. In pot experiment, the nodule number in the strain NL-Z1 treatment group did not increase, but the total nodule weight increased by 152.63% compared with the control group, and the hydrolytic nitrogen content in potted soil increased by 15.28%, and the value of bioactive enzymes increased from 45 to 121, increased by 169% (see table 4).

TABLE 4

| Experimental | Nitrogenase |
|---|---|
| treatment | activity |
| CK | 121 + 2.1 |
| NL-Z1 | 45 + 2.6 |

It was suggested that strain NL-Z1 could promote nitrogen fixation of Indigofera pseudotinctoria Matsum. and make the symbiotic nitrogen fixation ability play an effective role. The available phosphorus of potted soil treated by the strain NL-Z1 was improved obviously, and the growth indexes of corresponding plants increased significantly, indicating that the two were closely related. The root growth and nodule of Indigofera pseudotinctoria Matsum. in the strain NL-Z1 treatment group were significantly improved compared with the sterile control group. The results showed that the nodulation and nitrogen fixation were improved and the nitrogen nutrition were effectively supplemented. Therefore, NL-Z1 can indirectly promote the nodulation and nitrogen fixation of Indigofera pseudotinctoria Matsum. by promoting the release of available phosphorus in soil, which can be used as a functional strain of microbial fertilizer to promote the growth.

### Embodiment 3

The effect of strains on microbial community composition was explored.

The collected samples were sent to Shanghai Majorbio Bio-pharm Technology Co., Ltd. for sequencing using IlluminaMiseq platform.

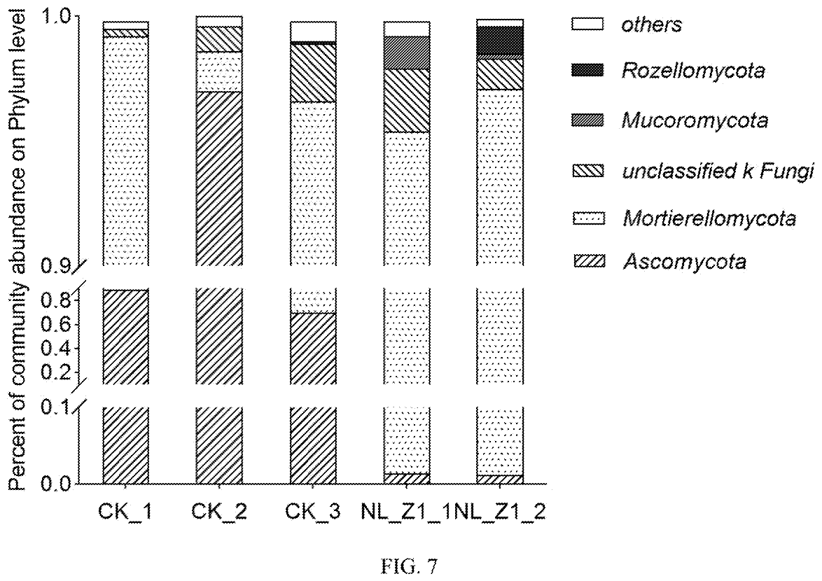

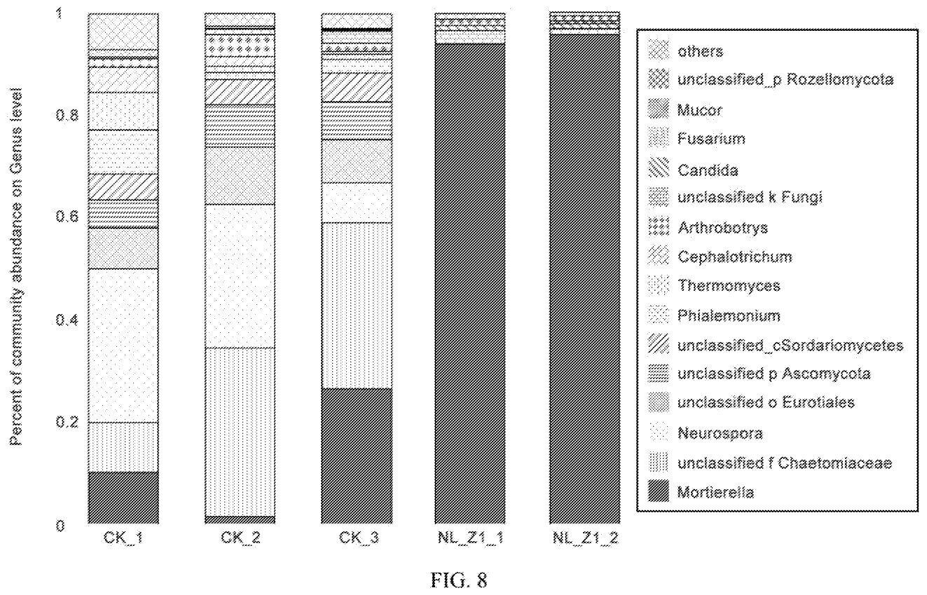

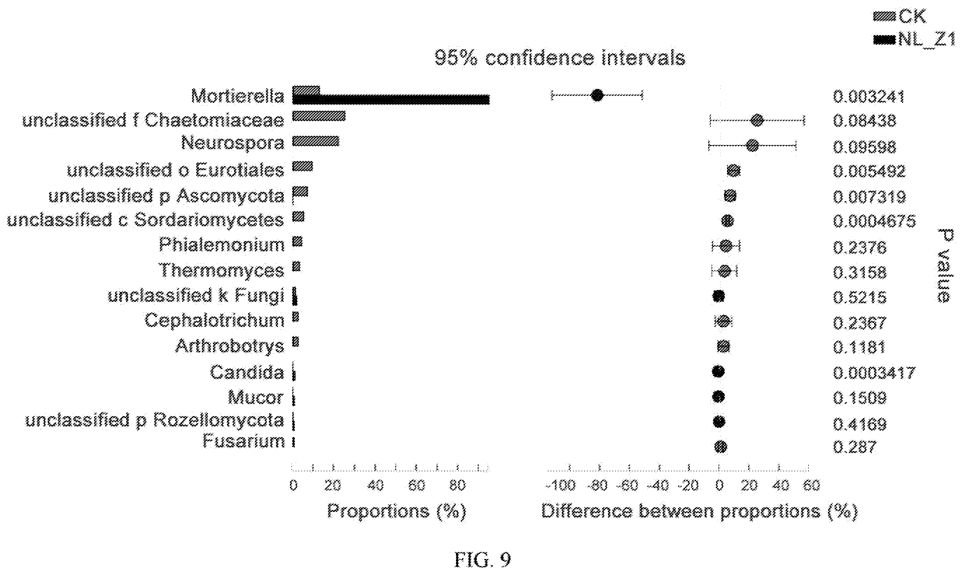

The microbial diversity of potted soil was detected by high throughput sequencing technology, and the microbial community composition of potted soil was analyzed. It can be seen from FIG. 7 that there was no difference in the microbial community composition between the control group and the potted soil treated with the strain NL-Z1 at the phylum level, mainly Ascomycota and Mortierellomycota. However, there were some differences in the relative abundance of microorganisms among the groups. After treatment with the strain NL-Z1, Ascomycota decreased from 85.32% to 1.32%, while Mortierellomycota increased from 12.81% to 94.95%, becoming a dominant phylum. The species composition of potted soil at genus level is shown in FIG. 8. The dominant genus in the strain NL-Z1 treatment group is Mortierella (circa 94.9%), while the relative abundance of Mortierella in control group is only 12.81%. Student's T test was used to test the significance of species difference at genus level, and the results are shown in FIG. 9. Mortierella was significantly different between the two groups (P<0.05).

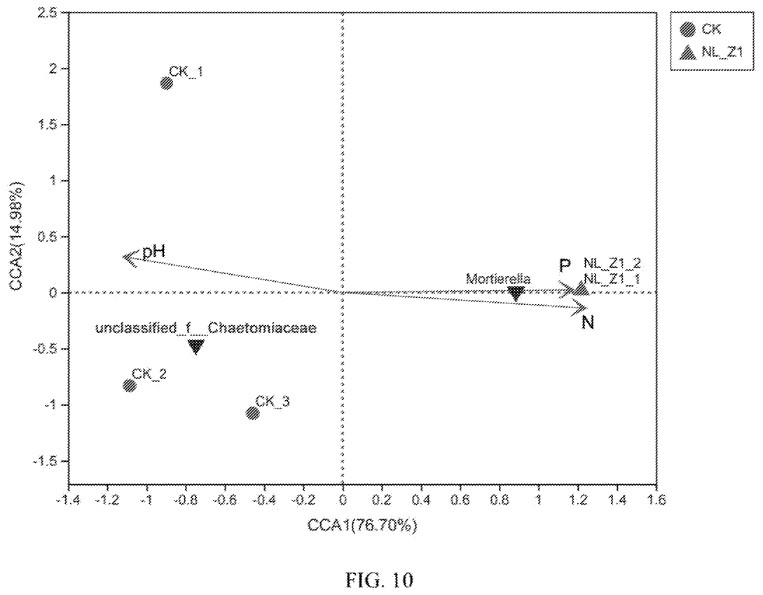

Canonical correlation analysis (CCA) was performed at genus level to reflect the relationship among environmental factors, flora and samples. The relationship is shown in FIG. 10 (axis1=76.7%, axis2=14.980%). The results showed that there was a positive correlation between available phosphorus and NL-Z1 community distribution (r2=0.85, P=0.25), a positive correlation between hydrolytic nitrogen and NL-Z1 community distribution (r2=0.95, P=0.16), and a negative correlation between pH and community distribution (r2=0.8, P=0.23). Among them, Mortierella had the greatest correlation with environmental factors.

The application of fungus NL-Z1 made the original dominant flora in soil evolve into Mortierellomycota at phylum level, and NL-Z1 did not belong to Mortierellomycota at taxonomic level, which indicated that the application of fungus NL-Z1 greatly changed the microbial community structure of soil. Mortierella was the dominant genus at genus level, and there were significant differences among groups, and it was detected by species difference analysis. Therefore, NL-Z1 agent could promote the increase of Mortierella in soil.

In addition, the addition of NL-Z1 agent from the extreme environment indirectly increased the relative abundance of Mortierella in the soil, and the amount of relative abundance increase and the promotion effect on plant body were better than that of direct application of Mortierella. In addition, the CCA analysis of environmental factors and fungal community showed that Mortierella was positively correlated with available phosphorus and hydrolyzed nitrogen, which indicated that NL-Z1 indirectly increased the release of nutrients in soil that were conducive to plant absorption and utilization by promoting the relative abundance of Mortierella. Therefore, NL-Z1 can be used as a kind of bacteria growth promoting agent, which can play an important role in planting legumes for slope protection in poor soil areas.

Each embodiment is described in a progressive manner, and each embodiment focuses on the differences from other embodiments. The same and similar parts of each embodiment can be referred to each other.

The above description of the disclosed embodiment enables those skilled in the art to realize or use the disclosure. Various modifications to these embodiments will be apparent to those skilled in the art, and the general principles defined herein can be implemented in other embodiments without departing from the spirit or scope of the present disclosure. Therefore, the present disclosure will not be limited to the embodiments shown herein, but will conform to the widest scope consistent with the principles and novel features disclosed herein.

1. (canceled)

2. A method for improving a relative abundance of microorganisms, wherein Penicillium simplicissimum sp. NL-Z1 is used as a bacterial fertilizer, and the deposit number is CCTCC NO: M2019999.

3. An application of Penicillium simplicissimum sp. NL-Z1 in promoting plant production of Indigofera pseudotinctoria Matsum., wherein Penicillium simplicissimum sp. NL-Z1 is used as a bacterial fertilizer, and the deposit number is CCTCC NO: M2019999.

4. An application of Penicillium simplicissimum sp. NL-Z1 in promoting plant nodule proliferation, and improving probiotic microorganisms and nutritional environments, wherein Penicillium simplicissimum sp. NL-Z1 is used as a bacterial fertilizer; a nodule weight of Indigofera pseudotinctoria Matsum. seedlings treated with Penicillium simplicissimum sp. NL-Z1 is increased; a total nodules weight is increased by at least 152.63%; the deposit number is CCTCC NO: M2019999; and the plant is Indigofera pseudotinctoria Matsum.

5. The application of claim 4, wherein after a treatment with strain NL-Z1, the abundance of probiotic microorganism population is increased; Ascomycota is decreased from 85.32% to at least 1.32%; and Mortierellomycota is increased from 12.81% to at least 94.95%, becoming a dominant phylum after the treatment with the strain NL-Z1.

6. The application of claim 5, wherein the Indigofera pseudotinctoria Matsum. seedlings treated with the strain NL-Z1 can promote the growth of plant roots and aboveground parts due to the increase of nodule mass, enzyme activity and the abundance of probiotic microorganism population; a root biomass is at least 1.60 g, increased by 33.33%; a root area is 246.53 cm², increased by at least 6.14%; a root volume is 2.12 cm³, increased by at least 23.98%; an aboveground biomass is 7.90 g, increased by 37.63%; an average ground diameter is 5.02 mm, increased by 10.33%; an average height of aboveground parts is at least 74.67 cm, increased by 36.58%; an average leaf area is at least 6.25 cm², increased by 24.50%; a hydrolytic nitrogen of rhizosphere soil increased by at least 15.28%; and a soil pH is decreased from 7.06 to 6.79.
